(19) United States
(12) Patent Application Publication
(10) Pub. No.: US 2016/0058409 A1
(43) Pub. Date: Mar. 3, 2016

(54) ULTRASONIC BLOOD PRESSURE MEASUREMENT APPARATUS AND BLOOD PRESSURE MEASUREMENT METHOD

(71) Applicant: SEIKO EPSON CORPORATION, Tokyo (JP)

(72) Inventor: Hiromitsu MIZUKAMI, Matsumoto-shi (JP)

(21) Appl. No.: 14/823,516

(22) Filed: Aug. 11, 2015

(30) Foreign Application Priority Data
Aug. 26, 2014 (JP) 2014-171267

(51) Int. Cl. A61B 8/04; A61B 8/02

(52) U.S. Cl. CPC A61B 8/04; A61B 8/02

(57) ABSTRACT

In an ultrasonic blood pressure measurement apparatus that measures a blood pressure by transmitting an ultrasonic wave toward a blood vessel and receiving a reflected wave, a storage unit stores a β blood-pressure calculation expression that is a first relationship between a vascular diameter of the blood vessel and a blood pressure; a pulse wave velocity calculation unit measures a pulse wave velocity of the blood vessel; a β blood-pressure calculation expression modifying unit calculates a modified β blood-pressure calculation expression that is a third relationship obtained by modifying the β blood-pressure calculation expression using the pulse wave velocity; a vascular diameter measurement unit configured to measure the vascular diameter of the blood vessel using the ultrasonic wave; and a temporary blood pressure calculation unit determines the blood pressure according to the modified β blood-pressure calculation expression.

### BACKGROUND

1. Technical Field

The present invention relates to an ultrasonic blood pressure measurement apparatus that measures a blood pressure using an ultrasonic wave.

2. Related Art

A technology for measuring the vascular diameter of the blood vessel of a subject using an ultrasonic wave and estimating a blood pressure from the vascular diameter is known as a technique for non-invasively measuring the blood pressure of the subject. For example, JP-A-2004-41382 discloses a method of obtaining a relationship between the vascular diameter and the blood pressure as a non-linear function, and calculating the blood pressure from the vascular diameter and a stiffness parameter β indicative of the stiffness of the blood vessel.

It is necessary to accurately measure the vascular diameter when the blood pressure is calculated from the vascular diameter. However, since the position of the blood vessel changes due to a body motion of the subject such as muscular contraction or a motion of a joint, accuracy in the measurement of the blood vessel may deteriorate. For example, due to a change in a relative positional relationship between the blood vessel and an ultrasonic probe, an ultrasonic signal used to measure the vascular diameter does not pass through the center of the blood vessel, or the intensity of reception of a reflected ultrasonic wave becomes weak, and thus it is not possible to measure the vascular diameter of the blood vessel.

### SUMMARY

An advantage of some aspects of the invention is that a blood pressure can be measured even if accuracy in the measurement of a vascular diameter using an ultrasonic wave deteriorates.

A first aspect of the invention is directed to an ultrasonic blood pressure measurement apparatus that measures a blood pressure by transmitting an ultrasonic wave toward a blood vessel and receiving a reflected wave, the apparatus including: a first relationship storage unit that stores a first relationship between a vascular diameter of the blood vessel and the blood pressure; a pulse wave velocity measurement unit that measures a pulse wave velocity of the blood vessel; a second relationship storage unit that stores a second relationship between the pulse wave velocity of the blood vessel and the blood pressure; and a blood pressure determination unit that measures the vascular diameter of the blood vessel using the ultrasonic wave, and determines the blood pressure based on the first relationship using the blood pressure corresponding to the measurement result of the pulse wave velocity measurement unit according to the second relationship, or measures the blood pressure based on the pulse wave velocity and the second relationship.

An eighth aspect of the invention is directed to a blood pressure measurement method of measuring a blood pressure by transmitting an ultrasonic wave toward a blood vessel and receiving a reflected wave, the method including: storing a first relationship between a vascular diameter of the blood vessel and the blood pressure; measuring a pulse wave velocity of the blood vessel; storing a second relationship between the pulse wave velocity and the blood pressure; and determining the blood pressure based on the vascular diameter of the blood vessel measured using the ultrasonic wave and the first relationship, or determining the blood pressure based on the pulse wave velocity and the second relationship.

According the first or eighth aspect of the invention, it is possible to measure the vascular diameter using the ultrasonic wave, and to determine the blood pressure according to the first relationship between the vascular diameter and the blood pressure, and it is possible to determine the blood pressure based on the measured pulse wave velocity and the second relationship between the pulse wave velocity and the blood pressure. Accordingly, for example, when accuracy in the measurement of the vascular diameter using the ultrasonic wave is high, it is possible to determine the blood pressure based on the first relationship, and when the accuracy of measurement is low, it is possible to determine the blood pressure based on the pulse wave velocity and the second relationship.

As a second aspect of the invention, the ultrasonic blood pressure measurement apparatus of the first aspect of the invention may be configured to further include a third relationship calculation unit that calculates a third relationship by modifying the first relationship using the blood pressure corresponding to the measurement result of the pulse wave velocity measurement unit according to the second relationship.

As a ninth aspect of the invention, the blood pressure measurement method of the eighth aspect of the invention may be configured to further include calculating a third relationship by modifying the first relationship using the blood pressure corresponding to the pulse wave velocity that is measured according to the second relationship.

According to the second or ninth aspect of the invention, the third relationship is calculated by modifying the first relationship using the blood pressure corresponding to the pulse wave velocity that is measured according to the second relationship.

As a third aspect of the invention, the ultrasonic blood pressure measurement apparatus of the first or second aspect of the invention may be configured such that the second relationship is represented by a linear expression.

As a tenth aspect of the invention, the blood pressure measurement method of the eighth or ninth aspect of the invention may be configured such that the second relationship is represented by a linear expression.

According to the third or tenth aspect of the invention, the second relationship is represented by a linear expression.

As a fourth aspect of the invention, the ultrasonic blood pressure measurement apparatus of any one of the first to third aspects of the invention may be configured such that when the measurement of the vascular diameter of the blood vessel using the ultrasonic wave satisfies predetermined reliability conditions, the blood pressure determination unit determines the blood pressure based on the measured vascular diameter and the first relationship, and the ultrasonic blood pressure measurement apparatus further includes: a database that stores the pulse wave velocity measured with the pulse wave velocity measurement unit, and the blood pressure which is determined by the blood pressure determination unit when the reliability conditions are satisfied; and a second relationship calculation unit that calculates the second relationship based on content stored on the database.

According to the fourth aspect of the invention, when the measurement of the vascular diameter of the blood vessel using the ultrasonic wave satisfies the predetermined reliability conditions, the database stores the pulse wave velocity and the blood pressure that is determined based on the measured vascular diameter and the first relationship, and the second relationship is calculated from the pulse wave velocity and the blood pressure which are stored in the database. Accordingly, it is possible to calculate the second relationship with relatively high reliability.

As a fifth aspect of the invention, the ultrasonic blood pressure measurement apparatus of the fourth aspect of the invention may be configured such that when the measurement of the vascular diameter of the blood vessel using the ultrasonic wave does not satisfy the reliability conditions, the blood pressure determination unit determines the blood pressure based on the pulse wave velocity and the second relationship.

According to the fifth aspect of the invention, when the measurement of the vascular diameter of the blood vessel using the ultrasonic wave does not satisfy the reliability conditions, the blood pressure is determined based on the pulse wave velocity and the second relationship.

As a sixth aspect of the invention, the ultrasonic blood pressure measurement apparatus of the fourth or fifth aspect of the invention may be configured such that the second relationship is a relationship between the pulse wave velocity and a systolic blood pressure, and the database stores the systolic blood pressure that is determined by the blood pressure determination unit.

According to the sixth aspect of the invention, the second relationship is a correlation between the pulse wave velocity and the systolic blood pressure. That is, it is possible to determine the systolic blood pressure based on the pulse wave velocity and the second relationship.

### BRIEF DESCRIPTION OF THE DRAWINGS

The invention will be described with reference to the accompanying drawings, wherein like numbers reference like elements.

FIG. 1 is a view illustrating the layout of an ultrasonic blood pressure measurement apparatus.

FIG. 2 is a view illustrating a measurement of a vascular diameter.

FIG. 3 is a graph illustrating a correlation between the vascular diameter and a blood pressure.

FIGS. 4A and 4B are graphs illustrating a calculation of a pulse wave velocity.

FIG. 5 is a graph illustrating a correlation between the pulse wave velocity and a systolic blood pressure.

FIG. 6 is a graph illustrating a change in the correlation between the vascular diameter and the blood pressure.

FIG. 7 is a diagram illustrating the functional configuration of the ultrasonic blood pressure measurement apparatus.

FIG. 8 is a flowchart illustrating a blood pressure measurement process.

### DESCRIPTION OF EXEMPLARY EMBODIMENTS

### Entire Configuration

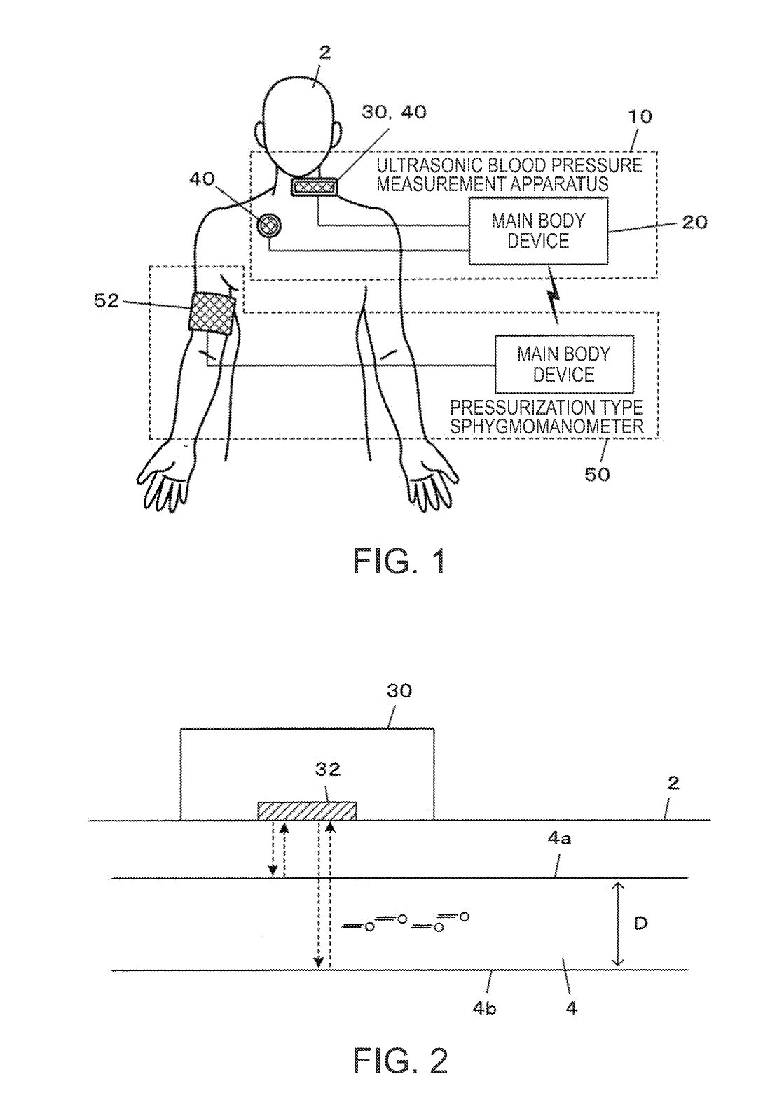

FIG. 1 is a view illustrating the layout of an ultrasonic blood pressure measurement apparatus 10 according to the invention. The ultrasonic blood pressure measurement apparatus 10 non-invasively measures the blood pressure of a subject using an ultrasonic wave, and includes a main body device 20; an ultrasonic probe 30 that is used for an ultrasonic measurement; and two electrocardiographic electrodes 40 that are used for an electrocardiographic measurement.

The ultrasonic probe 30 has an ultrasonic vibrator that transmits and receives an ultrasonic pulse signal or an ultrasonic burst signal with a frequency of several to several tens of MHz. For example, the ultrasonic probe 30 is attached to the left neck in such a way that the ultrasonic vibrator is positioned directly above the carotid artery of a subject 2. The term “directly above” referring to here is a term suitable for an operation manual for ease of understanding of an operation of the ultrasonic probe 30, and to be exact, the term “directly above” represents a positional relationship in which the carotid artery is positioned on an irradiation line of an ultrasonic wave that is irradiated from the ultrasonic vibrator arrayed in the ultrasonic probe 30.

One of the two electrocardiographic electrodes 40 is formed integrally with the ultrasonic probe 30, and the other electrocardiographic electrode 40 is attached to a region below the right clavicle of the subject 2.

The main body device 20 is wire-connected to the ultrasonic probe 30 and the electrocardiographic electrodes 40, and non-invasively measures the blood pressure of the subject 2 with the ultrasonic probe 30 and the electrocardiographic electrodes 40. Specifically, the ultrasonic probe 30 transmits an ultrasonic wave toward a blood vessel of the subject 2, a vascular diameter is measured based on a reflected wave signal which is received, and as a result, the blood pressure of the subject 2 can be calculated based on the measured vascular diameter. Hereinafter, a method of calculating the blood pressure from the vascular diameter is appropriately referred to as a “β method”.

An electrocardiographic waveform (ECG waveform) of the subject 2 is measured with the electrocardiographic electrodes 40, a pulse wave velocity PWV is calculated based on the measured electrocardiographic waveform, and as a result, the blood pressure of the subject 2 can be calculated based on the calculated pulse wave velocity. Hereinafter, a method of calculating the blood pressure from the pulse wave velocity PWV is appropriately referred to as a “PWV method”.

In the embodiment, typically, the blood pressure is calculated from the vascular diameter (the (3 method), and in contrast, when accuracy in the measurement of the vascular diameter using an ultrasonic wave has deteriorated or may deteriorate due to the body motion of the subject, a blood pressure calculation method switches to the method of calculating the blood pressure from the pulse wave velocity PWV (PWV method).

It is necessary to measure a blood pressure for calibration separately from the vascular diameter when the blood pressure is calculated based on the vascular diameter that is measured using an ultrasonic wave. In the embodiment, the blood pressure for calibration is measured with a pressurization type sphygmomanometer 50 that can communicate with the ultrasonic blood pressure measurement apparatus 10. The pressurization type sphygmomanometer 50 is configured such that a pressurization cuff 52 is wrapped around the upper arm of the subject 2, the blood pressure of the brachial artery of the subject 2 is measured, and a measured value is transmitted to the ultrasonic blood pressure measurement device 10. The cuff 52 is detached from the subject after the calibration is performed, and thereafter, the blood pressure of the subject 2 is non-invasively measured with the ultrasonic probe 30.

### Principle

### A. Measurement of Vascular Diameter

First, the measurement of the vascular diameter using an ultrasonic wave will be described. It is possible to calculate the vascular diameter based on positions of blood vessel walls relative to the ultrasonic probe 30, more specifically, based on depth positions of front wall and rear walls of the blood vessel.

FIG. 2 is a view illustrating the measurement of the positions of the blood vessel walls and the vascular diameter using an ultrasonic wave, and is a sectional view in a longitudinal direction of a blood vessel 4. As illustrated in FIG. 2, for measurement, the ultrasonic probe 30 is attached to the neck of the subject 2 in such a way that the ultrasonic vibrator 32 comes into close contact with the skin surface directly above the blood vessel 4. The ultrasonic vibrator 32 transmits an ultrasonic wave in a downward direction (in a depth direction) in FIG. 2. Characteristically, an ultrasonic wave is significantly reflected at the boundary surface of a medium. That is, when the blood vessel 4 is positioned directly below the ultrasonic vibrator 32, portions of the ultrasonic wave transmitted from the ultrasonic vibrator 32 are reflected by a front wall 4a and a rear wall 4b of the blood vessel 4, strong waves reflected by the front wall 4a and the rear wall 4b appear in a reflected wave signal of the ultrasonic vibrator 32. It is possible to measure the positions of the front wall 4a and the rear wall 4b based on an ultrasonic wave velocity and a difference in the time between a timing the ultrasonic wave is transmitted and a timing the reflected waves (which have been reflected by the front wall 4a and the rear wall 4b of the blood vessel 4) appear. Since the positions of the front wall 4a and the rear wall 4b are determined, a vascular diameter D can be obtained.

### B. Correlation Between Vascular Diameter and Blood Pressure

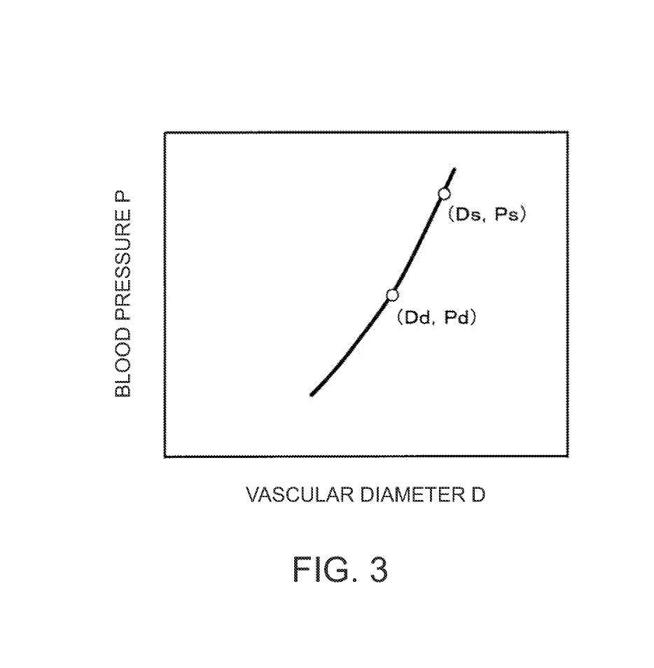

A description to be given hereinbelow relates to the calculation of a blood pressure based on the vascular diameter according to the β method. FIG. 3 is a graph illustrating a correlation between the vascular diameter D and a blood pressure P, and as illustrated in FIG. 3, the vascular diameter D and the blood pressure P have a non-linear relationship, and the non-linear relationship can be represented in Expression (1).

P=Pd×exp[β(D/Dd−1)] (1)

Here, β=In(Ps/Pd)/(Ds/Ds−1) (2)

In Expressions (1) and (2), “Pd” represents a diastolic blood pressure (the lowest blood pressure), “Dd” represents diastolic vascular diameter that is a vascular diameter when the blood pressure is the diastolic blood pressure, “Ps” represents a systolic blood pressure (the highest blood pressure), “Ds” represents a systolic vascular diameter that is a vascular diameter when the blood pressure is the systolic blood pressure, and “β” represents a vascular elasticity index value that is referred to as a stiffness parameter.

That is, it is possible to calculate the blood pressure P by substituting the vascular diameter D measured using the ultrasonic wave into Expression (1). Expression (1) is referred to as a β blood-pressure calculation expression. The β blood-pressure calculation expression is equivalent to a first relationship.

In order to calculate the blood pressure P from the vascular diameter D according to Expression (1), it is necessary to obtain the diastolic blood pressure Pd, the diastolic vascular diameter Dd, the systolic blood pressure Ps, the systolic vascular diameter Ds, and the stiffness parameter β, all of which are constants, and to perform calibration for Expression (1). In the embodiment, Expression (1) is defined by measuring the diastolic vascular diameter Dd and the systolic vascular diameter Ds using the ultrasonic probe 30, measuring the diastolic blood pressure Pd and the systolic blood pressure Ps using the pressurization type sphygmomanometer 50, and obtaining the stiffness parameter β based on the measured diastolic vascular diameter Dd, the measured systolic vascular diameter Ds, the measured diastolic blood pressure Pd, and the measured systolic blood pressure Ps according to Expression (2). The pressurization type sphygmomanometer 50 is not necessarily used to measure the blood pressures Pd and Ps for calibration, and another measuring means may be used for measurement.

### C. Pulse Wave Velocity

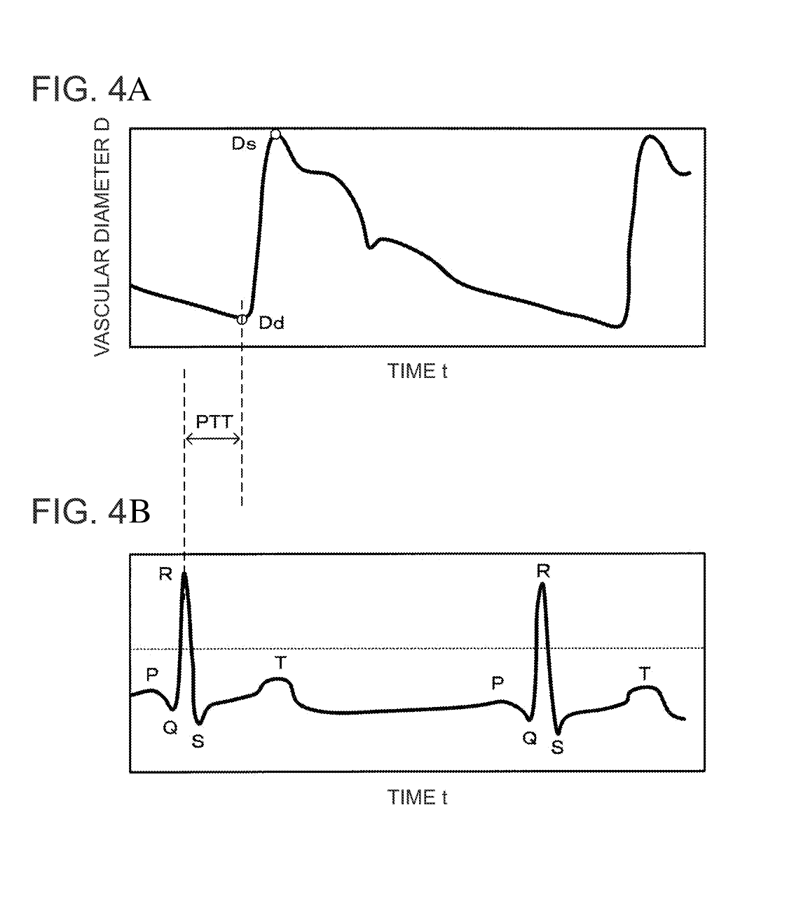

Hereinafter, the calculation of the pulse wave velocity will be described. FIGS. 4A and 4B are graphs illustrating the calculation of the pulse wave velocity PWV. FIG. 4A shows a waveform illustrating changes in the vascular diameter of the carotid artery over the period of one heartbeat, FIG. 4B illustrates an electrocardiographic waveform over the period of one heartbeat, and the time axe in FIG. 4A is aligned with the time axis in FIG. 4B.

The period of one heartbeat is made up of a systolic period and a diastolic period. In the systolic period, the vascular diameter is enlarged, and the blood vessel bulges, and in the diastolic period, the blood vessel is loose, and the vascular diameter is contracted, and returns to the original diameter. As illustrated in FIG. 4A, the vascular diameter is repeatedly changed for each heartbeat. FIG. 4B sequentially illustrates a P wave induced by the contraction of the atriums, a Q wave, a R wave, and an S wave induced by the contraction of the ventricles, and a T wave induced by the expansion of the ventricles in the electrocardiographic waveform.

An amount of time between a time the R wave of the electrocardiographic waveform in FIG. 4B reaches the peak and a time the vascular diameter reaches the minimum value in a vascular diameter change waveform in FIG. 4A represents a time (pulse propagation time) PTT that is required for a pulse induced by a heartbeat to reach the carotid artery in which measurement is performed using an ultrasonic wave. It is possible to calculate the pulse wave velocity PWV (=L/PTT) from the pulse propagation time PTT and a distance L between the attachment location of the electrocardiographic electrode 40 and the attachment location of the ultrasonic probe 30.

### D. Correlation Between Pulse Wave Velocity and Blood Pressure

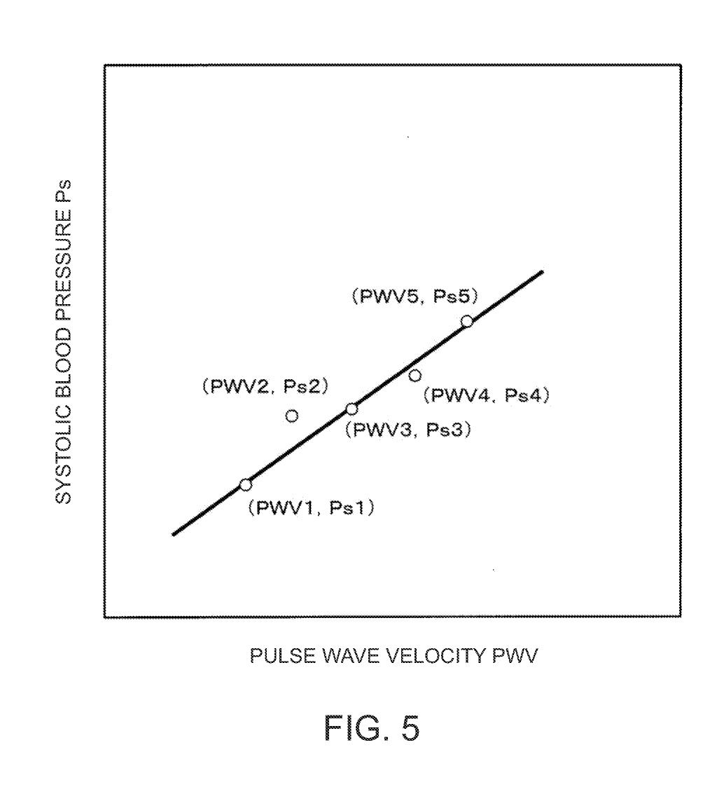

A description to be given hereinbelow relates to the calculation of a blood pressure based on the pulse wave velocity according to the PWV method. FIG. 5 is a graph illustrating a correlation between the pulse wave velocity PWV and the blood pressure Ps. As illustrated in FIG. 5, the pulse wave velocity PWV and the systolic blood pressure Ps have a linear relationship. This relationship can be represented by a linear expression as illustrated Expression (3).

Ps=A×PWV+B (3)

In Expression (3), A and B are constants. That is, it is possible to calculate the systolic blood pressure Ps by substituting the pulse wave velocity PWV (which is obtained based on the electrocardiographic waveform and the vascular diameter change waveform) into Expression (3). Expression (3) is referred to as a PWV blood-pressure calculation expression. The PWV blood-pressure calculation expression is equivalent to a second relationship.

For example, it is possible to derive Expression (3) using the method of least squares. In FIG. 5, only five items of sampling data are plotted, and it is possible to more accurately derive a relational expression by obtaining more items of sampling data.

A blood pressure obtained by the PWV method is the systolic blood pressure Ps. It is not possible to obtain the diastolic blood pressure Pd by the PWV method. The diastolic blood pressure Pd is calculated using an expression (hereinafter, which is referred to as a “modified β blood-pressure calculation expression”) that is a modification of the β blood-pressure calculation expression. The modified β blood-pressure calculation expression is equivalent to a third relationship.

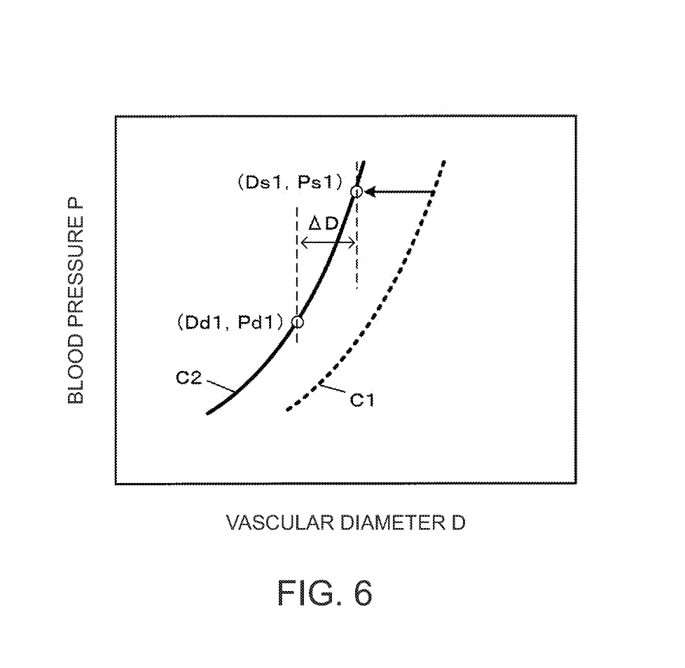

FIG. 6 is a graph illustrating the modified β blood-pressure calculation expression, and illustrates a correlation between the vascular diameter D and the blood pressure P. In FIG. 6, a curve C1 represents the β blood-pressure calculation expression that is not modified yet. When accuracy in the measurement of the vascular diameter using an ultrasonic wave is low due to the inclusion of a measurement error (diameter error) in each of a diastolic vascular diameter Dd1 and a systolic vascular diameter Ds1, the PWV method is used. Even though the accuracy in the measurement of the vascular diameter is low, it is possible to infer that a change ΔD (=Ds1−Dd1) in the vascular diameter, which is being tracked, is relatively accurate. That is, it is possible to infer that the measurement error contained in the diastolic vascular diameter Dd1 is equal to that contained in the systolic vascular diameter Ds1. The parallel transference of the curve C1 is performed in such a way that the parallel-transferred curve C1 passes through an intersection point between the systolic blood pressure Ps by the PWV method and the diastolic blood pressure Ds1 containing an error. A curve obtained in this manner is a curve C2.

The curve C2 is represented by Expression (4).

P=Ps1×exp[β(D/Ds1−1)] (4)

In Expression (4), Ps1, Ds1, and β are constants, Ps1 is the systolic blood pressure that is calculated by substituting the pulse wave velocity PWV into Expression (3), Ds1 is the diastolic vascular diameter that is measured using an ultrasonic wave, and β is the stiffness parameter that is determined by Expression (2). When the accuracy in the measurement of the vascular diameter is low, it is possible to calculate a blood pressure by defining Expression (4) and substituting the vascular diameter (containing an error) measured using an ultrasonic wave into Expression (4).

### Functional Configuration

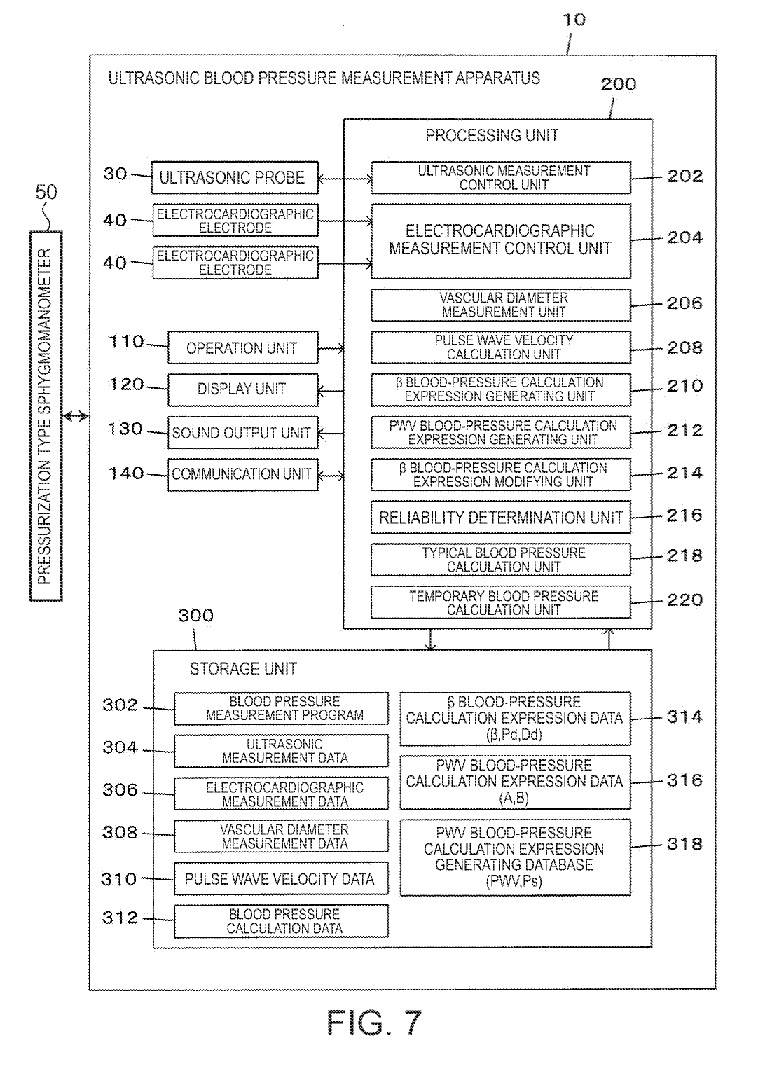

FIG. 7 is a diagram illustrating the functional configuration of the ultrasonic blood pressure measurement apparatus 10. In FIG. 7, the ultrasonic blood pressure measurement apparatus 10 is configured to include the ultrasonic probe 30; the electrocardiographic electrodes 40 and 40; an operation unit 110; a display unit 120; a sound output unit 130; a communication unit 140; a processing unit 200; and a storage unit 300.

The operation unit 110 is realized as input devices such as a button switch, a touch panel, and various sensors, and outputs an operation signal corresponding to a performed operation to the processing unit 200. The display unit 120 is realized as a display device such as a liquid crystal display (LCD), and displays various items of information in response to a display signal from the processing unit 200. The sound output unit 130 is realized as a sound output device such as a speaker, and outputs various sounds based on a sound signal from the processing unit 200. The communication unit 140 is realized as a wireless communication device such as a wireless local area network (wireless LAN) or Bluetooth (trademark), and communicates with an external device (mainly, the pressurization type sphygmomanometer 50).

The processing unit 200 is realized as electronic components such as a micro-processor (for example, a central processing unit (CPU), or a digital signal processor (DSP)), an application specific integrated circuit (AISC), and an integrated circuit (IC) memory, and controls an operation of the ultrasonic blood pressure measurement apparatus 10 by executing various computational processes based on programs and data stored in the storage unit 300, an operation signal from the operation unit 110, and the like. The processing unit 200 has an ultrasonic measurement control unit 202; an electrocardiographic measurement control unit 204; a vascular diameter measurement unit 206; a pulse wave velocity calculation unit 208; a β blood-pressure calculation expression generating unit 210; a PWV blood-pressure calculation expression generating unit 212; a β blood-pressure calculation expression modifying unit 214; a reliability determination unit 216; a typical blood pressure calculation unit 218; and a temporary blood pressure calculation unit 220.

The ultrasonic measurement control unit 202 controls the ultrasonic probe 30 such that the ultrasonic probe 30 transmits and receives an ultrasonic wave. Specifically, the ultrasonic measurement control unit 202 causes the ultrasonic probe 30 to transmit an ultrasonic wave over a predetermined period of transmission time. The ultrasonic measurement control unit 202 amplifies reflected ultrasonic wave signals that are received by the ultrasonic probe 30. Ultrasonic measurement data 304 for each of an A mode, a B mode, and an M mode is generated based on the reflected wave signal received by the ultrasonic probe 30.

The electrocardiographic measurement control unit 204 measures the electrical activity of the heart, that is, electrical changes over the period of a heartbeat. Specifically, the electrocardiographic measurement control unit 204 amplifies an electric potential difference between the two electrocardiographic electrodes 40, and converts the electric potential difference into a digital signal. The electrical activity of the heart measured with the electrocardiographic measurement control unit 204 is stored as electrocardiographic measurement data 306.

The vascular diameter measurement unit 206 calculates the vascular diameter based on the reflected ultrasonic wave signals that are received by the ultrasonic probe 30. That is, based on the intensities of the received signals, the vascular diameter measurement unit 206 determines the reception of the waves reflected by the front wall and the rear wall of the blood vessel. The vascular diameter measurement unit 206 calculates the positions (depth positions) of the front wall and the rear wall based on the difference in the time between a timing the ultrasonic wave is transmitted and a timing the reflected waves (which have been reflected by the front wall and the rear wall) are received. The vascular diameter measurement unit 206 calculates the vascular diameter from the positions of the front wall and the rear wall (refer to FIG. 2).

The ultrasonic probe 30 continues to transmit an ultrasonic wave and to receive a reflected wave, and the vascular diameter is repeatedly calculated every predetermined time (for example, at a time interval of approximately several to several tens of milliseconds, that is, substantially in real time). Accordingly, it is possible to obtain a waveform (refer to FIG. 4A) indicative of changes in the vascular diameter. The vascular diameters calculated by the vascular diameter measurement unit 206 are stored as vascular diameter measurement data 308 while being associated with measurement times.

The pulse wave velocity calculation unit 208 calculates the pulse wave velocity PWV from the vascular diameter measured with the vascular diameter measurement unit 206, and the electrocardiographic waveform measured with the electrocardiographic measurement control unit 204. That is, the pulse propagation time PTT is calculated as an amount of time between a time the peak of the R wave of the electrocardiographic waveform (which is measured with the electrocardiographic measurement control unit 204) appears and a time the minimum value of the vascular diameter appears in the vascular diameter change waveform that is measured with the vascular diameter measurement unit 206. The pulse wave velocity calculation unit 208 calculates the pulse wave velocity PWV (=L/PTT) from the pulse propagation time PTT, and the predetermined distance L between the ultrasonic probe 30 and the electrocardiographic electrode 40 (refer to FIG. 4). The pulse wave velocity calculated by the pulse wave velocity calculation unit 208 is stored as pulse wave velocity data 310.

The β blood-pressure calculation expression generating unit 210 generates the β blood-pressure calculation expression that is used to calculate the blood pressure P from the vascular diameter D. That is, Expression (1) indicative of the correlation between the vascular diameter D and the blood pressure P is defined by calculating the stiffness parameter β from the systolic blood pressure Ps and the diastolic blood pressure Pd (which are measured with the pressurization type sphygmomanometer 50), and the systolic vascular diameter Ds and the diastolic vascular diameter Dd (which are measured with the vascular diameter measurement 206) according to Expression (2).

It takes several to several tens of seconds to measure the blood pressure with the pressurization type sphygmomanometer 50. In parallel to the measurement of the blood pressure with the pressurization type sphygmomanometer 50, the systolic vascular diameter Ds and the diastolic vascular diameter Dd are obtained based on the vascular diameters which are calculated by the vascular diameter measurement unit 206. That is, the maximum value and the minimum value of the vascular diameter are detected for each heartbeat, the maximum value is regarded as the systolic vascular diameter Ds and the minimum value is regarded as the diastolic vascular diameter Dd.

The β blood-pressure calculation expression generated by the β blood-pressure calculation expression generating unit 210 is stored as β blood-pressure calculation expression data 314. Specifically, the β blood-pressure calculation expression data 314 contains the values of the parameter β, Ds, Pd, and Dd which are used to define the β blood-pressure calculation expression (Expression (1)).

The PWM blood-pressure calculation expression generating unit 212 generates the PWW blood-pressure calculation expression that is used to calculate the systolic blood pressure Ps from the pulse wave velocity PWV. That is, according to the method of least squares, the PWM blood-pressure calculation expression generating unit 212 generates a linear approximate expression based on sampling data of correlations (PWV, Ps) between the multiple pulse wave velocities PWV and the multiple systolic blood pressures Ps which are stored as a PWV blood-pressure calculation expression generating database 318. The PWV blood-pressure calculation expression generated by the PWV blood-pressure calculation expression generating unit 212 is stored as PWV blood-pressure calculation expression data 316. Specifically, the PWV blood-pressure calculation expression data 316 contains the values of parameters A and B which are used to define the PWV blood-pressure calculation expression (Expression (3)).

The correlation (PWV, Ps) between the pulse wave velocity PWV and the systolic blood pressure Ps, which are stored as the PWV blood-pressure calculation expression generating database 318, is a value that is calculated when the reliability determination unit 216 determines that reliability conditions are satisfied, which will be described later.

The β blood-pressure calculation expression modifying unit 214 generates the modified β blood-pressure calculation expression by modifying the β blood-pressure calculation expression that is generated by the β blood-pressure calculation expression generating unit 210. That is, the constants Pd and Dd in the β blood-pressure calculation expression are replaced with the systolic blood pressure Ps1 (which is calculated by substituting the pulse wave velocity PWV calculated by the pulse wave velocity calculation unit 208 into the PWV blood-pressure calculation expression generated by the PWV blood-pressure calculation expression generating unit 212) and the systolic vascular diameter Ds1 that is measured with the vascular diameter measurement unit 206. As a result, the modified β blood-pressure calculation expression is obtained. The β blood-pressure calculation expression modifying unit is equivalent to a temporary correlation calculation unit.

The reliability determination unit 216 determines whether the accuracy of the vascular diameter measured with the vascular diameter measurement unit 206 satisfies predetermined reliability conditions. The term “the reliability conditions being satisfied” represents that the accuracy in the measurement of the vascular diameter using an ultrasonic wave is good, and specifically, the term “the reliability conditions being satisfied” represents that the level of reception (signal intensity) of an ultrasonic wave reflected by the vascular intima is greater than or equal to a predetermined level.

When the reliability determination unit 216 determines that the reliability conditions are satisfied, the typical blood pressure calculation unit 218 calculates the blood pressure from the vascular diameter that is measured using an ultrasonic wave. For example, the blood pressure is determined by substituting the vascular diameter (which is measured with the vascular diameter measurement unit 206) into the β blood-pressure calculation expression that is generated by the β blood-pressure calculation expression generating unit 210. The typical blood pressure calculation unit 218 is equivalent to a blood pressure determination unit. When it is necessary to obtain only the systolic blood pressure Ps and the diastolic blood pressure Pd, it is possible to determine the systolic blood pressure Ps and the diastolic blood pressure Pd by substituting the maximum vascular diameter and the minimum vascular diameter for one heartbeat into the systolic vascular diameter Ds and the diastolic vascular diameter Dd, respectively, of the β blood-pressure calculation expression. Since the typical blood pressure calculation unit 218 works as the blood pressure determination unit, the typical blood pressure calculation unit 218 can be referred to as a functional unit equivalent to the blood pressure determination unit. The blood pressures Ps and Pd calculated by the typical blood pressure calculation unit 218 are stored as blood pressure calculation data 312 while being associated with measurement times.

When the reliability determination unit 216 determines that the reliability conditions are not satisfied, but it is possible to measure the change ΔD in the vascular diameter which is being tracked, and to determine that the change ΔD is substantially constant, the temporary blood pressure calculation unit 220 calculates the blood pressure from the pulse wave velocity. That is, for each heartbeat, the pulse wave velocity PWV calculated by the pulse wave velocity calculation unit 208 is substituted into the PWV blood-pressure calculation expression that is generated by the PWV blood-pressure calculation expression generating unit 212, and thus the systolic blood pressure Ps is calculated. Subsequently, the diastolic blood pressure Dd, which is the minimum value of the vascular diameter measured with the vascular diameter measurement unit 206, is substituted into the modified β blood-pressure calculation expression that is modified by the β blood-pressure calculation expression modifying unit 214, and thus the diastolic blood pressure Pd is calculated. Since the temporary blood pressure calculation unit 220 works as the blood pressure determination unit, the temporary blood pressure calculation unit 220 can be referred to as a functional unit equivalent to the blood pressure determination unit. The blood pressures Ps and Pd calculated by the temporary blood pressure calculation unit 220 are stored as the blood pressure calculation data 312 while being associated with measurement times.

The storage unit 300 is realized as storage devices such as a read only memory (ROM), a random access memory (RAM), and a hard disk, stores the programs and data which are used by the processing unit 200 so as to control the ultrasonic blood pressure measurement apparatus 10 in an integrated manner, is used as a working area of the processing unit 200, and temporarily stores results of computations performed by the processing unit 200, operation data from the operation unit 110, and the like. In the embodiment, the storage unit 300 stores a blood pressure measurement program 302; the ultrasonic measurement data 304; the electrocardiographic measurement data 306; the vascular diameter measurement data 308; the pulse wave velocity data 310; the blood pressure calculation data 312; the β blood-pressure calculation expression data 314; the PWV blood-pressure calculation expression data 316; and the PWV blood-pressure calculation expression generating database 318. The storage unit 300 is equivalent to a first correlation storage unit and a second correlation storage unit.

### Flow of Process

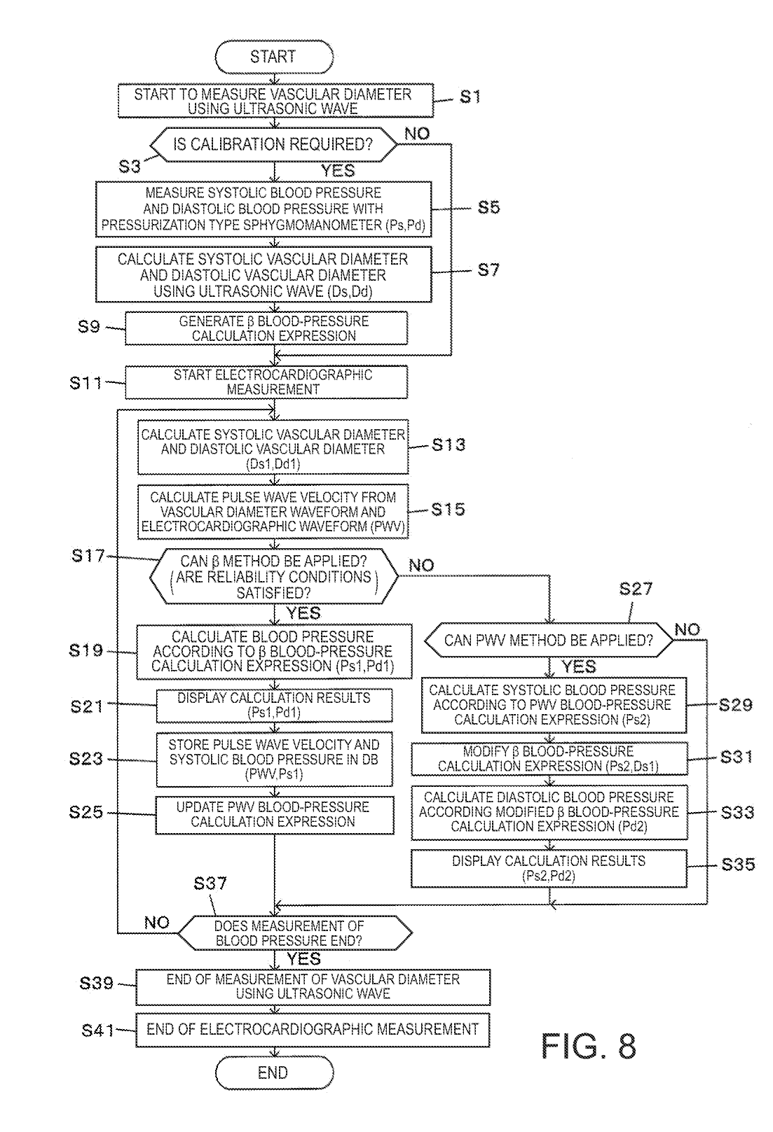

FIG. 8 is a flowchart illustrating the flow of a blood pressure measurement process. This process is a process that the processing unit 200 executes according to the blood pressure measurement program 302, and starts when there is an external instruction instructing to start the measurement of a blood pressure.

First, the ultrasonic measurement control unit 202 starts to perform ultrasonic measurement such that the ultrasonic probe 30 transmits and receives an ultrasonic wave, and the vascular diameter measurement unit 206 starts to measure a vascular diameter based on a reflected ultrasonic wave signal which is received (step S1).

Subsequently, the processing unit 200 determines whether calibration of the β blood-pressure calculation expression is required. For example, when the blood pressure of the subject 2 is measured with this apparatus for the first time, or when a predetermined time has elapsed since the last calibration is performed, the processing unit 200 determined that the calibration is required.

When it is determined that the calibration is required (YES: step S3), the display unit 120 displays a message instructing that the subject 2 wraps the cuff 52 around their upper arm, and measure the blood pressure with the pressurization type sphygmomanometer 50, measurement of the blood pressure of the subject 2 with the pressurization type sphygmomanometer 50 starts, and the maximum blood pressure (systolic blood pressure) Ps and the minimum blood pressure (diastolic blood pressure) Pd are measured (step S5). The vascular diameter measurement unit 206 calculates the systolic vascular diameter Ds and the diastolic vascular diameter Dd from the measured vascular diameter (step S7). When the measurement of the blood pressure with the pressurization type sphygmomanometer 50 ends, the β blood-pressure calculation expression generating unit 210 obtains the stiffness parameter β based on the vascular diameter Ds and Dd which are measured using the ultrasonic wave, and the blood pressures Ps and Pd which are measured with the pressurization type sphygmomanometer 50, and the β blood-pressure calculation expression generating unit 210 generates Expression (1) representative of the correlation between the vascular diameter and the blood pressure (step S9). These steps described up to now relates to the calibration.

Subsequently, the electrocardiographic measurement control unit 204 starts electrocardiographic measurement (step S11). The vascular diameter measurement unit 206 calculates the systolic vascular diameter Ds1 and the diastolic vascular diameter Dd1 from the measured vascular diameters for each heartbeat (step S13). The pulse wave velocity calculation unit 208 calculates the pulse wave velocity PWV from the measured vascular diameter waveform and the measured electrocardiographic waveform (step S15).

Subsequently, the reliability determination unit 216 determines whether the blood pressure can be calculated according to the β method. When the reliability conditions are satisfied, it is determined that the β method can be applied. When the β method can be applied (YES: step S17), the typical blood pressure calculation unit 218 calculates the systolic blood pressure Ps1 and a diastolic blood pressure Pd1 by substituting the systolic vascular diameter Ds1 and the diastolic vascular diameter Dd1 (which are calculated by the typical blood pressure calculation unit 218) into the β blood-pressure calculation expression (step S19). The calculated blood pressures Ps1_{—} and Pd1 along with the calculation method (in this case, the “β method”) are displayed (step S21). The typical blood pressure calculation unit 218 stores new sampling data, in which the calculated pulse wave velocity PWV is associated with the calculated systolic blood pressure Ps1, as the PWV blood-pressure calculation expression generating database 318 (step S23).

In contrast, when the β method cannot be applied (NO: step S17), it is determined whether the blood pressure can be measured according to the PWV method. When the change ΔD in the vascular diameter can be measured by a phase difference tracking method, it is determined that the PWV method can be applied.

When the PWV method can be applied (YES: step S27), the temporary blood pressure calculation unit 220 calculates a systolic blood pressure Ps2 by substituting the calculated systolic vascular diameter Ds1 into the PWV blood-pressure calculation expression (step S29). Subsequently, the β blood-pressure calculation expression modifying unit 214 generates the modified β blood-pressure calculation expression by modifying the β blood-pressure calculation expression using the calculated systolic vascular diameter Ds1 and the calculated systolic blood pressure Ps2 (step S31).

Subsequently, the temporary blood pressure calculation unit 220 calculates a diastolic blood pressure Pd2 by substituting the calculated diastolic vascular diameter Dd1 into the generated modified β blood-pressure calculation expression (step S33). The calculated blood pressures Ps2 and Pd2 along with the calculation method (in this case, the “PWV method) are displayed (step S35).

Thereafter, the processing unit 200 determines whether the measurement of the blood pressure ends due to an external instruction or the like, and when the measurement of the blood pressure has not ended (NO: step S37), the process returns to step S13. When the measurement of the blood pressure has ended (YES: step S37), the ultrasonic measurement control unit 202 ends the ultrasonic measurement, and the vascular diameter measurement unit 206 ends the measurement of the vascular diameter (step S39), and the electrocardiographic measurement control unit 204 ends the electrocardiographic measurement (step S41). The blood pressure measurement process ends with the completion of the aforementioned steps.

### Effects

When the accuracy in the measurement of the vascular diameter using an ultrasonic wave is low, the ultrasonic blood pressure measurement apparatus 10 of the embodiment can calculate the blood pressure using the pulse wave velocity PWV and the modified β blood-pressure calculation expression (Expression (4)) that is a modification of the 3 blood-pressure calculation expression (Expression (1)).

### Modification Example

An embodiment of the invention is not limited to the aforementioned embodiment, and an appropriate modification can be made to the embodiment insofar as the modification does not depart from the purport of the invention.

### A. Pulse Wave Velocity PWV

For example, the systolic blood pressure Ps may be calculated using the pulse propagation time PTT instead of the pulse wave velocity PWV. In this case, a correlation between the pulse propagation time PTT and the systolic blood pressure Ps is determined instead of Expression (3) representative of the correlation between the pulse wave velocity PWV and the systolic blood pressure Ps, and the systolic blood pressure Ps is calculated from the this correlation and the pulse propagation time PTT.

### B. Electrocardiographic Electrode 40

In the embodiment, one of the two electrocardiographic electrodes 40 and 40 is formed integrally with the ultrasonic probe 30; however, one of the two electrocardiographic electrodes 40 and 40 may not be formed integrally with the ultrasonic probe 30. In this case, the two electrocardiographic electrodes 40 and 40 are formed separately from the ultrasonic probe 30.

### C. PWV Blood-Pressure Calculation Expression

Expression (3) representative of the correlation between the pulse wave velocity PWV and the systolic blood pressure Ps is determined in terms of a linear function (linear expression) (refer to FIG. 5); however, Expression (3) may be determined in terms of a non-linear function (approximate curve).

### D. PWV Blood-Pressure Calculation Expression

The PWV blood-pressure calculation expression is determined by the correlation between the pulse wave velocity PWV and the systolic blood pressure Ps; however, the PWV blood-pressure calculation expression may be determined by a correlation between the pulse wave velocity PWV and the diastolic blood pressure Pd or an average blood pressure instead of the systolic blood pressure Ps.

### E. Target Blood Vessel for Measurement

The carotid artery is a target blood vessel for the ultrasonic measurement; however, the target blood vessel for measurement may be the brachial artery, the radial artery, the femoral artery, the subclavian artery, the main artery, or the like.

The entire disclosure of Japanese Patent Application No. 2014-171267 filed on Aug. 26, 2014 is expressly incorporated by reference herein.

What is claimed is:

1. An ultrasonic blood pressure measurement apparatus that measures a blood pressure by transmitting an ultrasonic wave toward a blood vessel and receiving a reflected wave, the apparatus comprising:
a first relationship storage unit that stores a first relationship between a vascular diameter of the blood vessel and the blood pressure;
a second relationship storage unit that stores a second relationship between a pulse wave velocity of the blood vessel and the blood pressure; and
a blood pressure determination unit that has a first blood pressure calculation unit configured to determine the blood pressure according to the first relationship based on the vascular diameter of the blood vessel which is measured using the ultrasonic wave, and a second blood pressure calculation unit configured to determine the blood pressure according to the second relationship based on the pulse wave velocity of the blood vessel which is measured using the ultrasonic wave.

2. The ultrasonic blood pressure measurement apparatus according to claim 1, further comprising,
a third relationship calculation unit that calculates a third relationship by modifying the first relationship using the blood pressure corresponding to the pulse wave velocity based on the second relationship.

3. The ultrasonic blood pressure measurement apparatus according to claim 1,
wherein the second relationship is represented by a linear expression.

4. The ultrasonic blood pressure measurement apparatus according to claim 1,
wherein when the measurement of the vascular diameter of the blood vessel using the ultrasonic wave satisfies predetermined reliability conditions, the blood pressure determination unit determines the blood pressure based on the measured vascular diameter and the first relationship,
wherein the ultrasonic blood pressure measurement apparatus further includes:
a database that stores the pulse wave velocity measured with a pulse wave velocity measurement unit, and the blood pressure which is determined by the blood pressure determination unit when the reliability conditions are satisfied; and
a second relationship calculation unit that calculates the second relationship based on content stored in the database.

5. The ultrasonic blood pressure measurement apparatus according to claim 4,
wherein when the measurement of the vascular diameter of the blood vessel using the ultrasonic wave does not satisfy the reliability conditions, the blood pressure determination unit determines the blood pressure based on the pulse wave velocity and the second relationship.

6. The ultrasonic blood pressure measurement apparatus according to claim 4,
wherein the second relationship is a relationship between the pulse wave velocity and a systolic blood pressure, and
wherein the database stores the systolic blood pressure that is determined by the blood pressure determination unit.

7. The ultrasonic blood pressure measurement apparatus according to claim 1, further comprising:
a pulse wave velocity measurement unit that measures the pulse wave velocity of the blood vessel.

8. A blood pressure measurement method of measuring a blood pressure by transmitting an ultrasonic wave toward a blood vessel and receiving a reflected wave, the method comprising:
storing a first relationship between a vascular diameter of the blood vessel and the blood pressure;
storing a second relationship between a pulse wave velocity of the blood vessel and the blood pressure; and
selecting either one of determining the blood pressure based on the vascular diameter of the blood vessel measured using the ultrasonic wave and the first relationship, and determining the blood pressure based on the pulse wave velocity of the blood vessel measured using the ultrasonic wave and the second relationship, based on predetermined reliability conditions.

9. The blood pressure measurement method according to claim 8, the method further comprising:
calculating a third relationship by modifying the first relationship using the blood pressure corresponding to the pulse wave velocity that is measured according to the second relationship.

10. The blood pressure measurement method according to claim 8,
wherein the second relationship is represented by a linear expression.
